(19) United States
(12) Patent Application Publication
(10) Pub. No.: US 2023/0277830 A1
(43) Pub. Date: Sep. 7, 2023

(54) Hand-held device for locally piercing a skin

(71) Applicant: MT.DERM GmbH, Berlin (DE)

(72) Inventor: Dirk Scherkowski, Greenwich, CT (US)

(21) Appl. No.: 18/178,962

(22) Filed: Mar. 6, 2023

(30) Foreign Application Priority Data
Mar. 7, 2022 (EP) 22160485.3

(51) Int. Cl. A61M 37/00

(52) U.S. Cl. CPC A61M 37/0076; A61M 2210/04

(57) ABSTRACT

A hand-held device for local piercing of skin includes: a housing; a drive unit within the housing adapted to provide a driving force; piercing means within the housing having at least one piercing needle disposed on a needle carrier; and coupling means within the housing and connecting the drive unit to the piercing means such that the driving force is transmissible to the piercing means to repeatedly move the needle carrier with the at least one piercing needle between a forward and a rearward position during operation. A lever mechanism receives a drive stroke provided by the drive device via an input of the lever mechanism on the drive side when the driving force is transmitted, to convert the drive stroke into a working stroke which is different from the drive stroke and to output the working stroke to the piercing device via an output of the lever mechanism.

The invention relates to a hand-held device for locally piercing a skin.

### CROSS-REFERENCE TO RELATED APPLICATIONS

The present application claims priority to European Patent Application No. 22160485.3 filed on Mar. 7, 2022, which is hereby incorporated by reference in its respective entirety.

### BACKGROUND

Such a hand-held device is used to locally pierce a human skin or an animal skin, for example to make a tattoo or for medical or cosmetic applications. For example, local skin piercing can be used to introduce a dye into upper layers of the skin, for example to create a tattoo or permanent makeup. But also the insertion of medical or cosmetic substances can be provided.

Usual hand-held devices have a housing in which a drive device is arranged, for example an electric motor, which in operation provides a mechanically generated driving force which is transmitted via a coupling device to a piercing device in the housing, so that the piercing device is moved forwards and backwards, the piercing of the skin regularly taking place at the end of the forward movement and the piercing needles of the piercing device used for this purpose being pulled out of the skin again by means of the backward movement. At least in the extended position, needle tips of the piercing needle(s) are arranged outside a front housing opening of the housing so that they can be pierced into the skin. The depth of penetration into the skin depends, among other things, on the length of the protrusion of the piercing needles relative to the front housing opening and the size of the working stroke performed by the piercing needles when they are moved into the extended (front) position.

### SUMMARY

The object of the invention is to provide a hand-held device for local skin piercing with improved operating characteristics.

For the solution, a hand-held device for local piercing of a skin according to claim 1 is provided. Embodiments are the subject of dependent claims.

According to one aspect, there is provided a hand-held device for locally piercing a skin, comprising: a housing; a drive unit disposed in the housing and adapted to provide a driving force; piercing means disposed in the housing and including at least one piercing needle disposed on a needle carrier; and coupling means disposed in the housing and connecting the a drive unit to the piercing means such that the driving force is transmissible to the piercing means to repeatedly move the needle carrier with the at least one piercing needle between a forward and a rearward position during operation. The coupling device has a lever mechanism which is set up to receive, on the drive side, a drive stroke provided by the drive device via an input of the lever mechanism when the driving force is transmitted, to convert the drive stroke into a working stroke which is different from the drive stroke, and to deliver the working stroke to the piercing device via an output of the lever mechanism.

The lever mechanism provided in the coupling device makes it possible to selectively change the drive stroke provided on the drive side in order to transmit the driving force from the drive device to the piercing device, in that the lever mechanism converts the drive stroke into the working stroke, which is different from the drive stroke. In this way, a working stroke can be provided for the operation of the piercing device that is not limited to the drive stroke provided on the drive side. Depending on the desired design of the handheld unit, the drive stroke provided on the drive side can be changed in relation to the application and converted into the working stroke.

The lever mechanism can be arranged to convert the drive stroke with a drive stroke length into the working stroke with a working stroke length that is different from the drive stroke length. In this embodiment, it is provided that the drive stroke is extended by means of the lever mechanism, which enables, for example, the provision of a larger needle protrusion of the piercing needle tips relative to the front housing opening, thereby optionally a larger piercing depth during operation of the hand-held device when piercing the skin locally.

The working stroke length can be greater than the drive stroke length. Alternatively, the working stroke length can be smaller than the drive stroke length. This makes it possible to use a drive device also in handheld devices that are to provide a working stroke in operation that is shorter or smaller than the drive stroke provided by the drive device.

In various applications, it can be provided that the lever mechanism is used to reverse or invert the direction of movement of the original drive stroke for the working stroke.

The lever mechanism may be formed with at least two cooperating levers. The at least two interacting levers can be pivotably mounted on a bearing component, for example in a respective associated bearing, wherein the respective bearing is associated with limiting means which limit a pivoting or rotary movement of the respective lever during operation of the lever mechanism during the stroke conversion.

The lever mechanism may be designed as a rigid lever mechanism between the input and the output. A rigid lever mechanism means in particular that elements or components of the lever mechanism, for example the at least two interacting levers, are fixedly connected to one another, for example a distance between the respective bearing of levers of the lever mechanism is fixed at a constant level during operation. In particular, the rigid lever mechanism may comprise or consist of flexure hinges, Due to the fixed connection formed by means of the rigid lever mechanism, a (force) transmission characteristic formed by means of the lever mechanism between the input and the output can be constant at all times during operation for moving the needle carrier with the at least one piercing needle back and forth.

The needle carrier with the at least one piercing needle may be displaceable during displacement from the rear to the front position against a biasing force provided by a retraction device. The biasing force may be configured to at least partially provide a total biasing force to be applied for displacement of the needle carrier with the at least one piercing needle from the front position to the rear position. The needle carrier with the at least one piercing needle is displaced back to the rear position with the aid of the total biasing force after displacement to the front position, which is an extended position with respect to the front housing opening for the piercing needle tip(s). The biasing force may in particular be introduced into a shaft of the piercing device rigidly coupled to the needle carrier.

This displacement between the forward and rearward positions is performed repeatedly at a repetition frequency during operation, so that the hand-held device can be used to locally pierce a skin multiple times. In one embodiment, the selective biasing force provided by the retraction device may fully correspond to the total biasing force. The retraction device may comprise one or more restoring elements formed, for example, by one spring element or a plurality of spring elements. In this case, it can be provided that the spring elements are stretched when the needle carrier with the at least one piercing needle is displaced into the forward position and thus develop the biasing force. The biasing force may then be coupled to the needle carrier or another element of the moving parts, for example the piercing device, for moving the needle carrier back (retraction). To form a proportional biasing force or a total biasing force, for example, the lever mechanism can also be set up, in particular based on the action of a torque around the pivot point of one or more levers.

As an alternative to the provision of a retraction device, the hand-held device can be designed without such a (optionally supplementary) retraction device in such a way that, with the inclusion of the lever mechanism, a (particularly releasable) rigid (non-positive) coupling is formed between the drive device and the piercing device, so that—in addition to the driving force for displacement into the front position (push force)—the biasing force for displacement from the front into the rear position is also provided directly by the drive device and is transmitted to the stay device via the rigid (non-positive) connection (pull force).

In the housing, a sealing membrane can sealingly separate a front housing chamber from a rear housing chamber. The sealing membrane may for example be realized as a sealing diaphragm or as a sealing lip. The front housing space may open to the front housing opening. In particular, the sealing membrane prevents body fluid that may enter the front housing space through the front housing opening when the hand-held device is used for local skin piercing from then also entering the rear housing space. The rear housing space may at least partially receive the drive device. The piercing device can be at least partially arranged in the front housing space, in particular the needle carrier with the at least one piercing needle.

The retraction device may be formed with the sealing membrane, in particular the sealing diaphragm, such that the biasing force is at least partially formed when one or more elastic membrane sections of the sealing membrane are stretched from the rear position to the front position during displacement of the needle carrier with the at least one piercing needle. When the needle carrier with the at least one piercing needle is displaced to the forward position during operation, the elastic membrane sections of the sealing membrane, in particular of the sealing diaphragm, stretch so that the restoring or restoring force is provided by the sealing membrane itself. In this case, a coupling section of the sealing membrane is fixedly connected, in particular in a non-slipping manner, to the needle carrier, either directly or indirectly via an intermediate component, so that the elastic membrane sections are stretched when displaced to the forward position and the sealing membrane, in particular the sealing diaphragm, can then couple its biasing force to the needle carrier to support its displacement from the forward to the rearward position or to cause it on its own.

In an alternative embodiment, the sealing membrane may comprise a sealing lip. In particular, the sealing lip engages a component in slidingly manner moveable between the rear position and the front position. A sealing lip may slidingly and sealingly engage a component of the hand-held device, in particular of the piercing device, more particularly a shaft thereof. A sealing lip slidingly engaging a component may be configured not to exert a biasing force onto said component. A sealing lip may be fixed to the needle carrier, wherein a coupling section of the sealing lip may be fixedly connected, in particular in a non-slipping manner, to the needle carrier, and a gliding section of the sealing lip engages another component, such as the lever mechanism. Alternatively, the sealing lip may be fixed to the lever mechanism, wherein a coupling section of the sealing lip is fixedly connected, in particular in a non-slipping manner, to the lever mechanism, and a gliding section of the sealing lip engages another component, such as the needle carrier. In an embodiment comprising a sealing lip, additional biasing means, such as a spring, in particular a compression spring or a tension spring, shall be provided for providing a biasing force for the retraction device.

The lever mechanism can be arranged in the rear housing space. In this way, the lever mechanism is sealed off from the front housing space by means of the sealing membrane. The lever mechanism can be arranged at least partially in the front housing space. In an alternative embodiment, the lever mechanism is arranged in a front housing space.

The needle carrier of the piercing device can be directly connected to the output of the lever mechanism. Alternatively, an indirect connection or coupling between the needle carrier and the lever mechanism can be formed via an intermediate component associated with the needle carrier, for example via one or more connecting components.

An output member, at which the output of the lever mechanism is formed, may be pivotally received in an associated receptacle on the needle carrier of the piercing device. For example, a lever portion of one of the levers of the lever mechanism extending substantially transverse to the longitudinal direction of the housing may be pivotally received in the associated receptacle on the needle carrier.

The housing can be made in several parts with housing parts that detachable from each other. The housing is designed in multiple parts with several housing parts that detachable from each other. The housing parts can comprise disposable and/or reusable modules. Disposable modules are designed for single use and are replaced after use of the hand-held skin piercer. Reusable modules, on the other hand, are used multiple times.

The housing parts that are detachable from each other may each house one or more of the functional components or devices of the handheld device. If two housing parts each have a functional component or device of the handheld device, coupling means may be provided to couple the two functional components when the housing parts are connected, and to decouple them when the housing parts are disconnected or released from each other. For example, this may involve coupling means for connecting the drive device in one housing part to a functional component receiving the drive motion or driving force in another housing part. The coupling means can, for example, have a plug-in, clamping and/or screw connection for coupling.

The coupling means may comprise a magnetic component configured for transferring the driving force from the drive unit to the piercing device. The coupling means may comprise a reception member for engaging a driving pin. A driving pin may be rotatable in an oscillating manner. The reception member may be moveably fixed to the front housing part. The driving pin may be moveably fixed to the rear housing part. The reception member may be configured to receive an oscillating rotational driving movement, in particular form a driving pin. The reception member may be coupled to the piercing device, in particular to the needle carrier. In particular, the reception member is coupled to the piercing device, in particular to the needle carrier, via the lever mechanism. The lever mechanism may be configured to transfer the driving force from the driving unit to the needle carrier. Optionally, the lever mechanism may be configured to transfer a biasing force from a biasing means to the needle carrier. It may be preferred that the lever mechanism transforms the rotational oscillatory movement from the driving unit into a corresponding oscillating linear movement of the needle carrier.

A handpiece can be formed with a rear housing part, in which the drive device is arranged. A skin-piercing module can be formed with a front housing part, in which the piercing device is arranged. The handpiece is gripped by the user's fingers during operation of the hand-held device to guide the handheld device, and may be formed as a multi-use module. The rear housing member may in turn comprise one or more partial housing members. The skin piercing module with the piercing device may be formed as a disposable module so that it is replaceably attached to another housing part, in particular the rear housing part. In one embodiment, a housing interior of the skin piercing exchange module is part of the front housing space, which is sealed off from the rear housing space by means of the sealing membrane.

The lever mechanism may be at least partially disposed in the skin piercing module.

The rear housing part can be a handpiece with drive device of an electric toothbrush. The drive unit of the rear housing part, in particular of the electric toothbrush, may in particular comprise a driving pin configured to move rotationally in an oscillating manner. Alternatively, the drive unit of the rear housing part, in particular of the electric toothbrush, may in particular comprise a driving member configured to move linearly in an oscillating manner. A linearly oscillating driving member may be configured to oscillate in a direction corresponding to, in particular parallel to or equal to, the axial direction of the rear housing part, and/or the axial direction of the front housing part. The handpiece includes the drive unit and is adapted to be releasably coupled to an attachment having a brushing means, so that—in addition to the skin brushing means—an electric toothbrush is provided. In this way, an article can be provided comprising the handpiece with the a drive unit of an electric toothbrush, as well as a skin-piercing module and a brush (changing) module with the brush means, each of which can be releasably coupled to the handpiece with the a drive unit, such that the driving force or movement provided by the a drive unit can be coupled to the respectively coupled module.

The lever mechanism can be arranged in the front housing part. The lever mechanism can be arranged in the skin piercing exchange module. Alternatively, the lever mechanism can be arranged in a coupling module that can be detachably connected to the handpiece of the electric toothbrush. The piercing device can then be arranged in the skin piercing change module, which is detachably arranged on the coupling module.

The hand-held unit can have an adjustment mechanism that is set up to adjust a needle protrusion or needle overhang, i.e. an overhang or protrusion of the needle tips in relation to a front housing edge or a front housing opening through which the piercing needles move when they are moved to the forward (extended) position. In the adjustment mechanism, a housing portion on the front module housing may be adjustable relative to the needle carrier and thus to the piercing needles. For example, after coupling the skin piercing module to the handpiece, the housing part may be connected (coupled) in the region of a rear housing section to a sleeve which is adjustable or displaceable in the longitudinal direction of the housing, such that adjustment or displacement of the sleeve in the longitudinal direction of the housing, for example by rotating the sleeve along a threaded bearing of the sleeve on the rear housing part, changes the relative position of the housing part with respect to the needle carrier with the piercing needles and thus with respect to the needle tips, so that a needle projection or needle protrusion of the needle tips relative to the front housing edge is thereby set.

In one embodiment, a piercing needle tip of the at least one piercing needle is arranged to protrude beyond a front housing opening, at least in the forward position. Alternatively, the piercing needle tip may be arranged behind the front housing opening in both the front and rear positions, wherein the piercing needle tip is arranged closer to the front housing opening in the front position than in the rear position, optionally in the region of the front housing opening.

It may be preferred that the housing comprises a front housing part and a rear housing part. The front housing part and the rear housing section part may be releasably coupled to each another. The front housing part may comprise a first casing for sealingly housing components and/or devices contained therein. The piercing device may in particular moveably fixed to the front housing part. The piercing device may be received within the front housing. It may be preferred to arrange the piercing device completely and constantly outside of the rear housing. Particularly, the piercing device is moveable with respect to the first casing so as to be at least partially or completely retractable into the first casing and/or so as to be at last partially or fully protrude beyond a front housing opening of the first casing. The lever mechanism may be received, in particular completely, within the front housing. It may be preferred to arrange the lever mechanism completely and constantly outside of the rear housing. The front housing part may in particular be realized as a piercing exchange module. The front housing part may be realized as a, preferably reusable or disposable, needle cartridge. The front housing part preferably comprises a skin-piercing module. The rear housing part may comprise a second casing for sealingly housing components and/or devices contained therein. Preferably the drive device is contained within the rear housing section. Particularly, the rear housing part may be realized as a handpiece. In a particular embodiment, the rear housing part can be a handpiece with drive device of an electric toothbrush. The drive device may be received within the rear housing. It may be preferred to arrange the drive device completely and constantly outside of the front housing.

While the present disclosure generally refers to skin piercing needles, the disclosure is to be understood to relate also to stimulating needles. Stimulating needles may have rounded tips. A rounded tip may have a tip radius of no less than 0.05 mm, in particular no less than 0.1 mm. The rounded tip may have a tip radius of no more than 0.5 mm, in particular no more than 0.25 mm, preferably no more than 0.1 mm. In a particular embodiment, the rounded tip may have a tip radius in the range between no less than 0.05 mm and no more than 0.08 mm. Stimulating needles may be configured not to pierce an animal skin and/or human skin.

### DESCRIPTION OF EMBODIMENT EXAMPLES

The patent or application file contain at least one drawing executed in color. Copies of the patent or patent application publication with color drawings(s) will be provided by the Office upon request and payment of the necessary fee.

In the following, further embodiments are explained with reference to figures of a drawing. Here show:

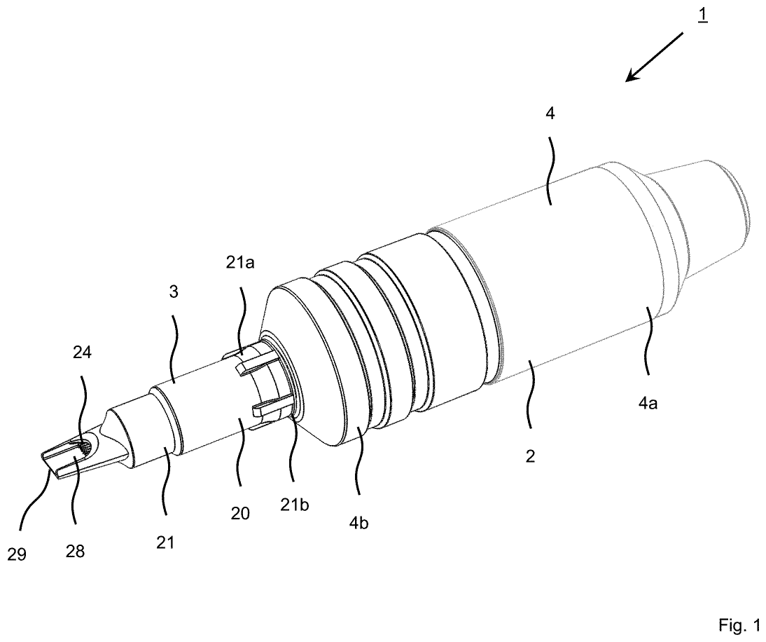

FIG. 1 schematic perspective view of a hand-held device for local skin piercing;

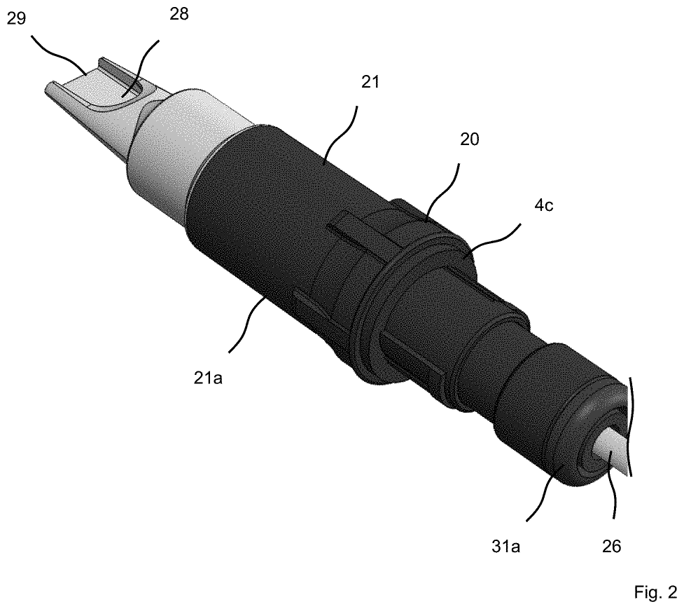

FIG. 2 schematic perspective view of a skin piercing module;

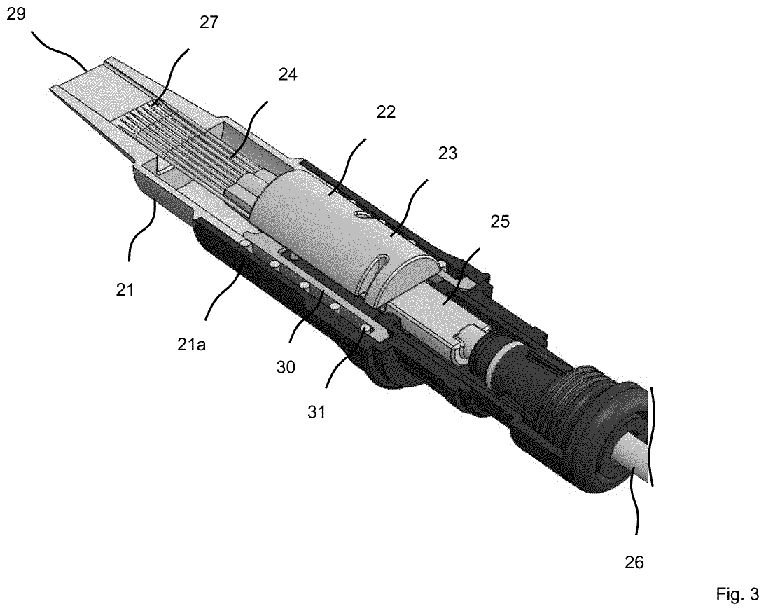

FIG. 3 schematic perspective view of the skin piercing module from FIG. 2 in section;

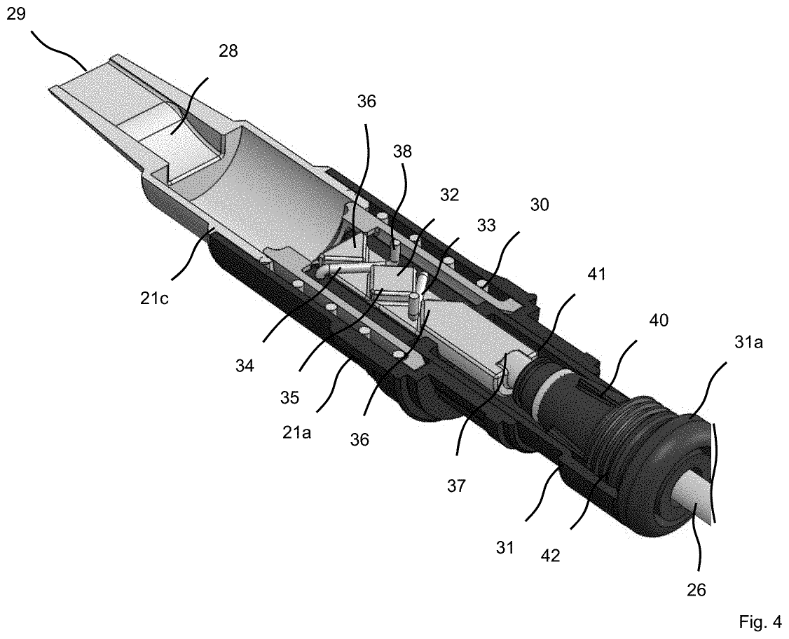

FIG. 4 another schematic perspective view of the skin piercing module from FIG. 2 in section;

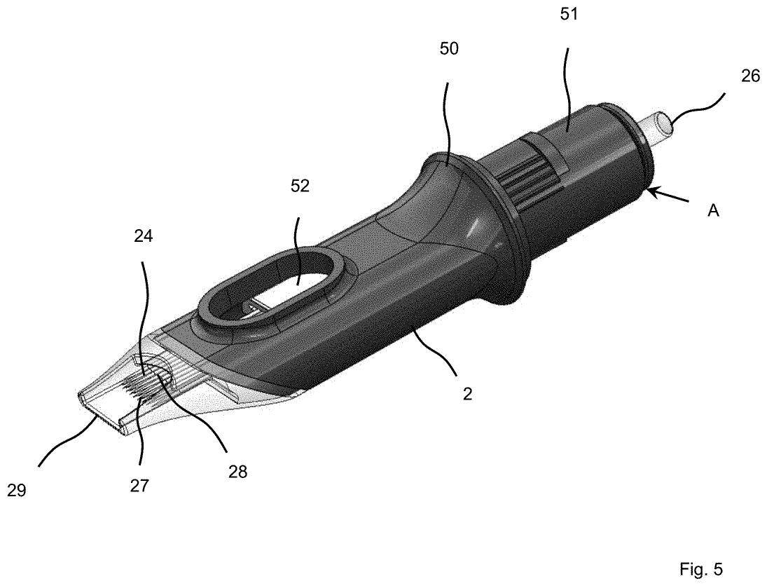

FIG. 5 schematic perspective view of another skin piercing module;

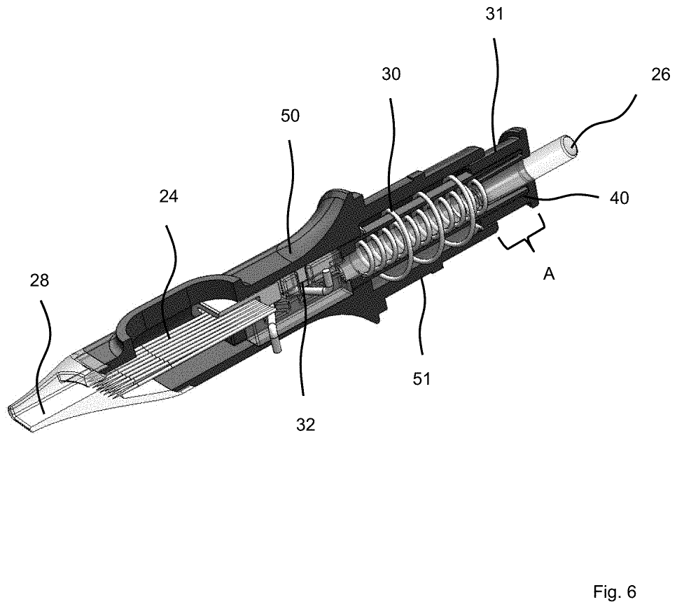

FIG. 6 schematic perspective view of the other skin piercing module of FIG. 5 partially in section;

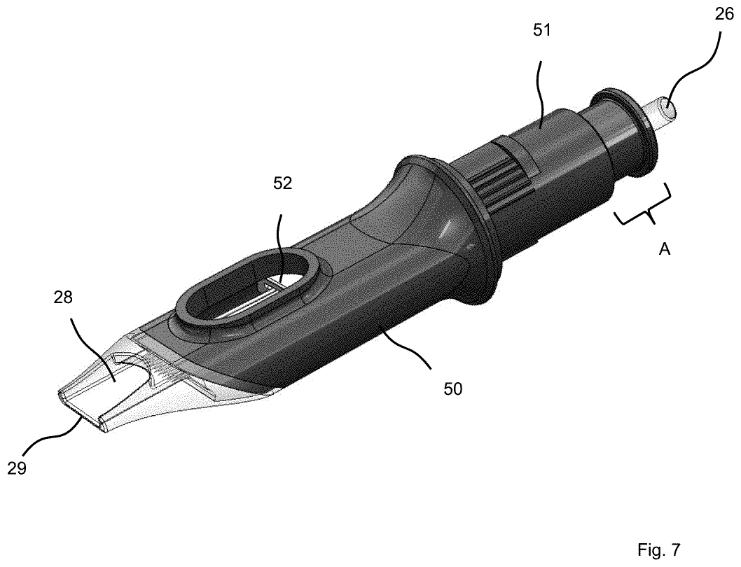

FIG. 7 another schematic perspective view of the other skin piercing module from FIG. 5, with piercing needles retracted further;

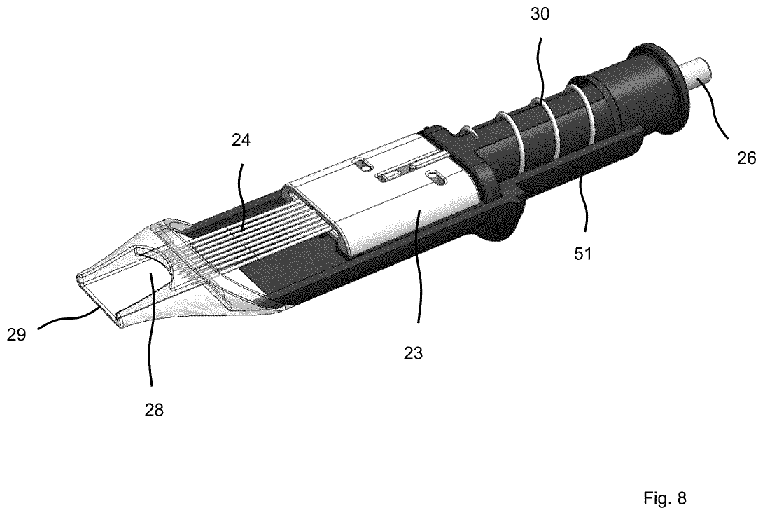

FIG. 8 another schematic perspective view of the other skin piercing module from FIG. 5 in section;

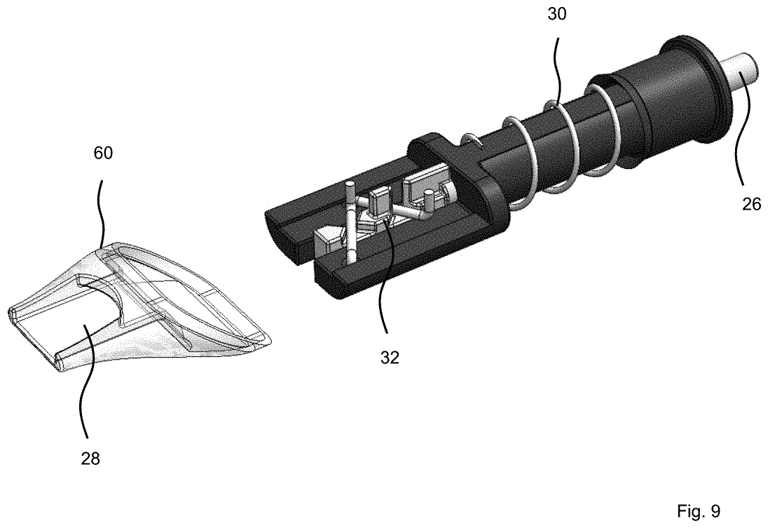

FIG. 9 schematic perspective view of elements of the other skin piercing module in FIG. 5;

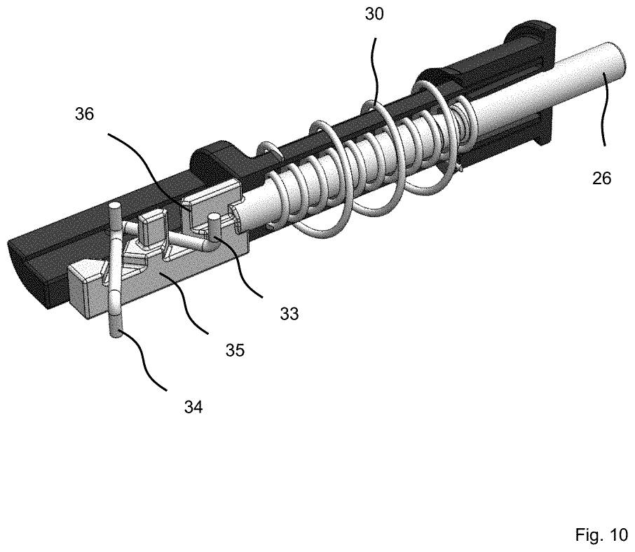

FIG. 10 another schematic perspective view of elements of the other skin piercing module from FIG. 5 in section;

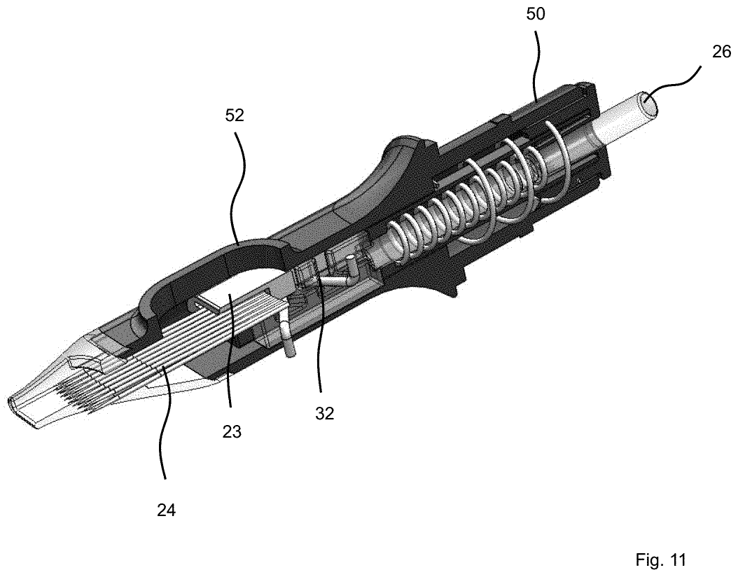

FIG. 11 schematic perspective view of the other skin piercing module of FIG. 5, with the piercing needles arranged in an intermediate position;

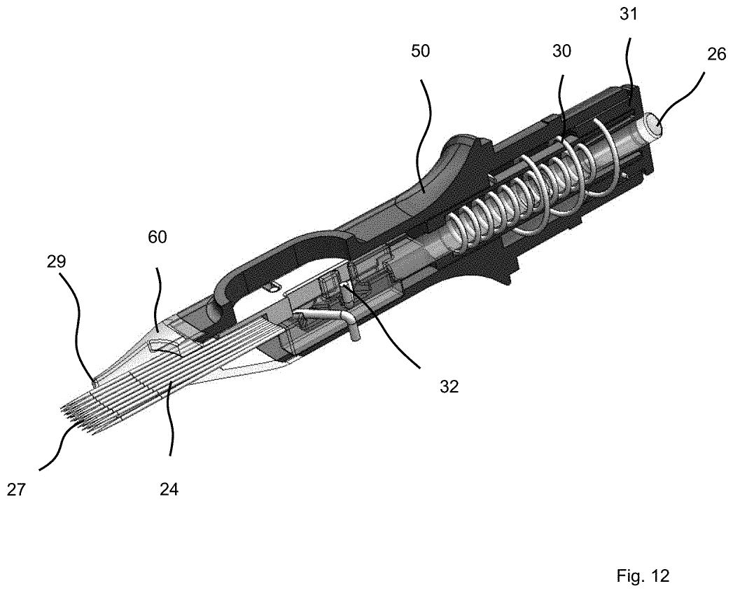

FIG. 12 schematic perspective view of the other skin piercing module of FIG. 5 in section, with the piercing needles arranged in an extended (forward) position;

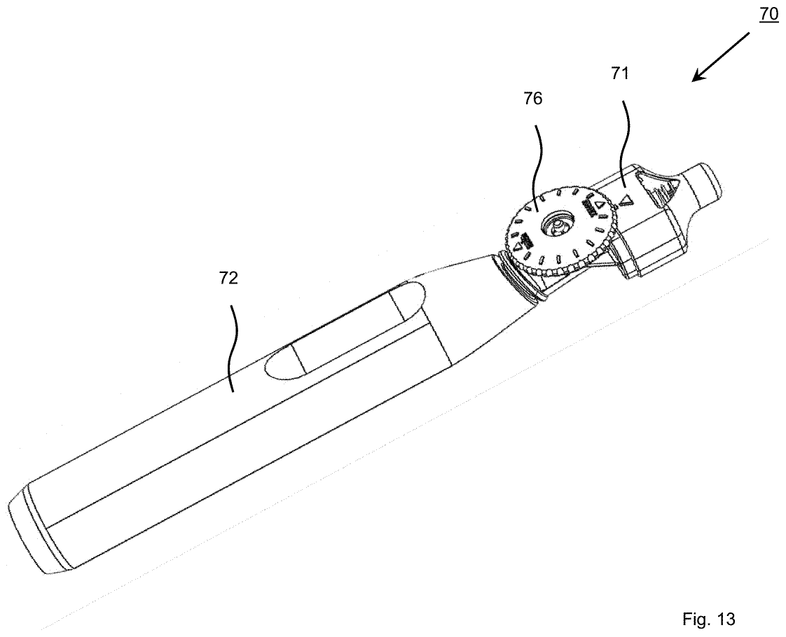

FIG. 13 schematic perspective view of a further embodiment of a hand-held device for locally brushing a skin with a handpiece of an electric toothbrush;

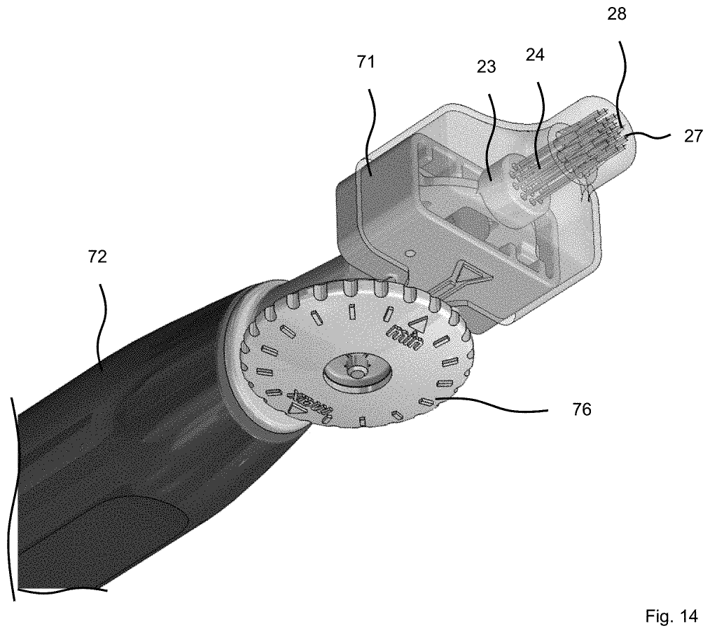

FIG. 14 schematic partial perspective view of the hand-held device of FIG. 13 and

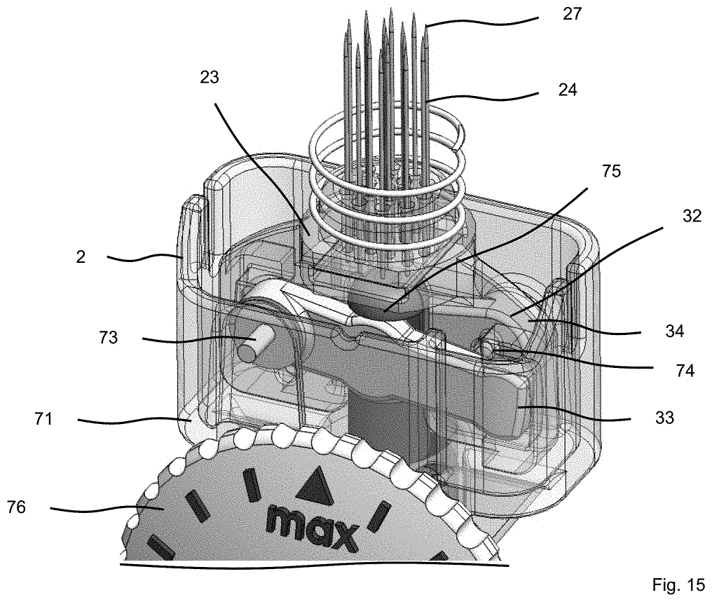

FIG. 15 schematic partial perspective view of the handheld unit from FIG. 13.

FIG. 1 shows a schematic perspective view of an embodiment of a hand-held device 1 for local skin piercing. The hand-held device 1 has a housing 2 with a front and a rear housing part 3, 4, which are detachably connected to one another in the embodiment example shown. The rear housing part 4 provides a handpiece 4a which the user grips with fingers during operation.

FIGS. 2 to 4 show schematic perspective views of an exchangeable skin piercing module (skin piercing exchange module) 20, which is formed with the front housing component 3 in the hand-held device 1 of FIG. 1.

A piercing device 22 is arranged in a module housing 21, in which piercing needles 24 are arranged on a needle carrier 23. The needle carrier 23 is connected to a module-side coupling device 25 in such a way that, during operation, a driving force provided via a coupling component 26, which is provided by a drive device in the housing 2, is transmitted to the needle carrier 23, so that the needle carrier 23 with the piercing needles 24 arranged thereon is moved back and forth in the longitudinal direction of the housing between a front and a rear position. FIG. 3 shows the needle carrier 23 with the piercing needles 24 in the rear position, in which needle tips 27 of the piercing needles 24 are arranged behind a front housing opening 28 (cf. FIG. 2) (retracted position). To pierce the skin, the needle carrier 23 with the piercing needles 24 is moved into a front position (cf. FIG. 12 below), in which the needle tips 27 protrude with respect to the front housing opening 28, in particular with respect to a front housing edge 29.

The needle carrier 23 is guided in the module housing 21 during back and forth movement in operation. A spring element 30 ensures that a housing sleeve 31 with a cap 31a is pressed against a bottom of a module receptacle on the handpiece 4a after docking and regardless of the setting of a needle protrusion of the piercing needle tip 27 with respect to the front housing opening 28 or the front housing edge 29.

After coupling the skin piercing module 20 to the handpiece, a housing part 21a on the module housing 21 is connected in the region of a rear housing section 21b to a sleeve 4b that can be adjusted or displaced in the longitudinal direction of the housing 2 (cf. FIG. 1) in such a way that an adjustment or displacement of the sleeve in the longitudinal direction of the housing 2, for example by means of rotating the sleeve 4b along a threaded bearing of the sleeve 4b on the rear housing section 4, changes the relative position of the housing section 21a with respect to the needle carrier 23 with the piercing needles 24 and thus with respect to the needle tips 27, so that a needle projection or needle protrusion of the needle tips 27 relative to the front housing edge 29 is set as a result.

The housing part 21a abuts a circumferential ledge 21c on the module housing 21, which forms a stop for the housing part 21a.

In the shown embodiment example, the displacement into the front position towards the front housing opening 28 takes place against a biasing force of a sealing membrane 40, which is stretched in the process (cf. FIG. 4). The sealing membrane 40 is then part of a retraction device which provides a biasing force, and at the same time seals a front housing space 41 (front side with respect to the sealing membrane 40 in the housing of the hand-held device) against a rear housing space 42 (rear side with respect to the sealing membrane 40 in the housing of the hand-held device, in the direction of the drive device). Also with respect to the module housing 21, the sealing membrane 40 divides it into a front housing space and a rear housing space within the module housing 21.

The sealing membrane made of an elastic material then also contributes to moving the needle carrier 23 with the piercing needles 24 back to the rear (retracted) position according to FIG. 3, after the needle carrier 23 with the piercing needles 24 has previously been displaced to the front position. In this way, the needle carrier 23 with the piercing needles 24 performs a back and forth movement in the longitudinal direction of the housing 2 during operation, in response to the coupled driving force, with a repetition frequency that can be varied, for example, with the aid of an electric motor of the drive device.

Referring to FIG. 4, a lever mechanism 32 is provided in the interchangeable skin cutting model as a component of a module-side part of the coupling device by means of which the driving force is transmitted to the needle carrier 23, which lever mechanism is formed in the embodiment example shown with a first lever 33 and a second lever 34, each of which is pivotally mounted on a bearing component 35. The bearing component 35 has limiting means 36 associated with the first and second levers 33, 34 and limiting their pivotal movement in operation. With the aid of the lever mechanism 32, a drive stroke which is transmitted via the coupling component 26 to an input 37 of the lever mechanism 32 is converted into a working stroke which is transmitted from the lever mechanism via an output 38 to the needle carrier 23 with the piercing needles 24, the working stroke being different from the drive stroke coupled to the lever mechanism 32, for example having an increased or reduced stroke length. In this way, it is possible to convert or convert the drive stroke received from the drive device into a working stroke specifically transmitted to the needle carrier 23 with the piercing needles 24, which then determines which path (stroke) the piercing needles 24, in particular their needle tips 27, execute during operation. For example, the stroke conversion can be used to influence the protrusion of the piercing needle tips 27 relative to the front housing edge 29, and thus the piercing depth during local piercing of the skin with the aid of the hand-held device 1.

In the example shown for the lever mechanism 32, this is designed to increase the stroke, which means that the decoupled working stroke is greater than the coupled drive stroke. The stroke length is increased. In alternative embodiments, the stroke length can remain the same or can be shortened by means of the lever mechanism 32.

In the embodiment shown, the biasing force provided by the sealing membrane 40 optionally supplements the lever mechanism 32 at least when moving the stay device back.

FIGS. 5 to 12 show schematic diagrams for an embodiment of another needle piercing module 50. The same reference signs are used in FIGS. 5 to 12 as in FIGS. 2 to 4 for identical features.

In FIGS. 5 to 12, a respectively set needle projection or needle protrusion, which the needle tips 27 assume in relation to the front edge of the housing 29 when the piercing device is moved to the front position (cf. FIG. 12), can be seen by means of a respectively set distance A (cf. for example FIGS. 5 to 7).

For this purpose, a housing part 51 is adjustable or displaceable after coupling to the handpiece 4a by means of the sleeve 4b formed thereon (cf. FIG. 1) relative to the needle carrier 23 with the piercing needles 24 and thus with respect to the needle tips 27. Here, FIG. 5 (as also FIG. 12) shows for the other needle piercing module 50 a position of the needle tips 27 with an adjusted maximum needle projection or needle protrusion (distance A minimum). FIG. 11 shows a sectional view of the other needle piercing module 50 in the position shown in FIG. 5.

FIG. 7 shows a minimum needle out or needle overhang with the piercing needles 24 retracted to the rear position. FIGS. 6, 8 to 10 show different sectional views of the other needle piercing module 50 with the position shown in FIG. 7.

If the other needle piercing module 50 is used for an insertion of a substance during local piercing of the skin, it is possible to insert the substance to be inserted via a module opening 52, so that it can reach the pierced skin in particular due to capillary forces along the piercing needles 24. In the embodiment of the needle piercing module 20 in FIGS. 2 to 4, the substance, for example a dye or a cosmetic/medical active ingredient, can be applied in the region of the front housing opening 28.

Referring to FIG. 9, a module tip 60 is detachably arranged on the module housing 21. The module tip 60 may be detachably arranged from the module housing 21 in unity with the piercing needles 24 or the needle carrier 23 (with piercing needles 24).

While the pins 24 are shown in particular in FIGS. 6 to 10 in a rear (retracted) position, FIG. 5 shows an intermediate position in which the pins 27 can be seen through the front housing opening 28 (see also FIG. 11). Finally, FIG. 12 shows the piercing needles 24 in a front (extended) position (maximum needle projection), in which the piercing needle tips 27 protrude from the front housing opening 28 protrusion well as the front housing edge 29. Hereby a needle protrusion is formed, which is characterized by a needle length with which the piercing needles 24 protrude with respect to the front housing edge 29.

In the examples in FIGS. 5 to 12, the lever mechanism 32 is also designed to increase the stroke.

FIG. 13 shows a schematic perspective view of another embodiment of a hand-held device 70, in which an attached needle piercing module 71 is detachably arranged on a handpiece 72, which is formed by a handpiece of an electric toothbrush with a drive device accommodated therein. FIGS. 14 and 15 each show a schematic partial perspective view of the hand-held device of FIG. 13. The same reference signs are used in FIGS. 13 to 15 as in FIGS. 1 to 12 for identical features.

The lever mechanism 32 is formed with the first and second levers 33, 34 formed in a crossed arrangement. The first and second levers 33, 34 are pivotally mounted to the module housing by means of a respective bearing 73, 74. The drive stroke is introduced to the lever mechanism 32 via a coupling component 75, and the converted working stroke is then transmitted to the needle carrier 23. In this way, the driving force provided by the handpiece of the electric toothbrush is transmitted to the needle carrier 23 with the positioning needles 24, with the stroke conversion or conversion taking place in this case.

An adjustment element 76, which in the example shown is formed with a rotary element, is arranged to adjust a needle protrusion or needle protrusion for the piercing needles 24 relative to the front housing opening 28 by rotating the rotary element. For this purpose, it is provided that the rotating element cooperates with an eccentric mechanism (not shown) in such a way that a position of a sleeve component 77, at which the front housing opening 28 is formed, can be changed relative to the piercing needle tips 27 in the extended (front) position of the piercing needles 24.

The features disclosed in the foregoing description, the claims, and the drawing may be significant, both individually and in any combination, for the realization of the various embodiments.

1. A hand-held device for local piercing of a skin, comprising:
a housing;
a drive device arranged in the housing and adapted to provide a driving force;
a piercing device arranged in the housing and having at least one piercing needle arranged on a needle carrier; and
a coupling device is arranged in the housing and connecting the drive device to the piercing device so that the driving force is transmitted to the piercing device in order to move the needle carrier with the at least one piercing needle repeatedly between a front and a rear position during operation;
wherein the coupling device has a lever mechanism which is set up to receive a drive stroke provided by the drive device via an input of the lever mechanism on the drive side when the driving force is transmitted, to convert the drive stroke into a working stroke which is different from the drive stroke, and to deliver the working stroke to the piercing device via an output of the lever mechanism.

2. The hand-held device according to claim 1, wherein the lever mechanism is arranged to convert the drive stroke having a drive stroke length into the working stroke having a working stroke length different from the drive stroke length.

3. The hand-held device according to claim 2, wherein the working stroke length is greater than the drive stroke length.

4. The hand-held device according to claim 2, wherein the working stroke length is smaller than the drive stroke length.

5. The hand-held device according to claim 1, wherein the lever mechanism is formed with at least two interacting levers.

6. The hand-held device according to claim 1, wherein the lever mechanism between the input and the output is designed as a rigid lever mechanism.

7. The hand-held device according to claim 1, wherein the needle carrier with the at least one piercing needle is displaceable during displacement from the rear position into the front position against a biasing force provided by a retraction device, and wherein the biasing force is arranged to provide, at least partially, a total biasing force to be applied for displacement of the needle carrier with the at least one piercing needle from the front position into the rear position.

8. The hand-held device according to claim 1, wherein a sealing membrane in the housing separates a front housing chamber from a rear housing chamber in a sealing manner.

9. The hand-held device according to claim 8, wherein the restoring means is formed with the sealing membrane such that the biasing force is formed when elastic membrane portions of the sealing membrane are stretched from the rear position to the front position when the needle carrier with the at least one piercing needle is displaced.

10. The hand-held device according to claim 8, wherein the sealing membrane comprises a sealing lip engaging a component in a sliding manner moveable between the rear position and the front position.

11. The hand-held device according to claim 1, wherein the needle carrier of the piercing device is directly connected to the output of the lever mechanism.

12. The hand-held device according to claim 11, wherein an output element, at which the output of the lever mechanism is formed, is pivotally mounted in an associated receptacle on the needle carrier of the piercing device.

13. The hand-held device according to claim 1, wherein the housing is configured comprising multiple pieces including housing parts detachable from one another.

14. The hand-held device according to claim 13, wherein a handpiece is formed with a rear housing part, in which the drive device is arranged, and a skin-piercing module is formed with a front housing part, in which the piercing device is arranged.

15. A hand-held device according to claim 14, wherein the rear housing part is a handpiece with a drive unit of an electric toothbrush.

16. A hand-held device according to claim 14, wherein the lever mechanism is arranged in the front housing part.

17. The hand-held device according to claim 8, wherein the lever mechanism is arranged in the rear housing space.
